(19) United States
(12) Patent Application Publication
(10) Pub. No.: US 2012/0322873 A1
(43) Pub. Date: Dec. 20, 2012

(54) USE OF A BUFFER WITH A BIOCIDE PRECURSOR

(75) Inventors: Jeffery M. Atkins, Aurora, IL (US); Amit Gupta, Aurora, IL (US); Charles J. Reedy, Chicago, IL (US); Barbara E. Moriarty, Palatine, IL (US)

(73) Assignee: Nalco Company, Naperville, IL (US)

(21) Appl. No.: 13/595,555

(22) Filed: Aug. 27, 2012

Related U.S. Application Data
(63) Continuation-in-part of application No. 12/979,806, filed on Dec. 28, 2010.

(51) Int. Cl. C07C 407/00; A01P 1/00; B01J 19/00; A01N 37/16

(52) U.S. Cl. 514/557; 562/4; 422/129

(57) ABSTRACT

Disclosed herein are biocidal compounds and compositions, methods of using said compounds and compositions, and systems and processes for their preparation. Particularly disclosed herein are in situ generated biocides stabilized with a buffer. The biocide may include in situ generated peracetic acid produced from a peroxygen source, an acyl group donor, and a buffer.

### CROSS-REFERENCE TO RELATED APPLICATION

This is a continuation-in-part of U.S. Ser. No. 12/979,806 filed Dec. 28, 2010, the contents of which is herein incorporated by reference.

### TECHNICAL FIELD

The present disclosure relates generally to biocides, and more particularly to in situ generated biocides stabilized with a buffer.

### BACKGROUND

Aqueous systems in industrial processes exist so that necessary chemical, mechanical and biological processes can be conducted to reach the desired outcome. Fouling, the presence or deposition of microbial populations in an aqueous system of concern, can occur even when the system is treated with the best water treatment programs currently available. Aqueous systems can become heavily fouled if microbial populations are not controlled. Fouling can have a negative impact on the industrial process utilizing the aqueous system, resulting in reduced heat transfer and higher associated energy costs. Sources of microbial contamination in industrial aqueous systems are numerous and diverse and may include, but are not limited to, air-borne contamination, water make-up, process leaks, process conditions or the process itself, and improperly cleaned equipment. Microbial populations can be very system specific and their metabolism can be significantly impacted by the system conditions.

Exopolymeric substances secreted from microorganisms aid in the formation of biofilms as the microbial communities develop on a surface. These biofilms are complex ecosystems that establish a means for concentrating nutrients and offer protection for growth. Biofilms can accelerate scale, corrosion, and other fouling processes. Not only do biofilms contribute to reduction of system efficiencies, but they also provide an excellent environment for microbial proliferation that can include pathogenic bacteria. It is therefore important that biofilms and other fouling processes be reduced to the greatest extent possible to maximize process efficiency and minimize the health-related risks from water-borne pathogens.

Several factors contribute to the problem of biological fouling and govern its extent. Water temperature, water pH, organic and inorganic nutrients, growth conditions such as aerobic or anaerobic conditions, and in some cases the presence or absence of sunlight, can play an important role.

There are several different methods for controlling microbial populations in an aqueous system. Broadly speaking, the microbial control methods can be physical (e.g., ultraviolet, thermal energy) or chemical in nature. Chemical microbial control methods can be further divided into oxidizing or non-oxidizing types. Both oxidizing and non-oxidizing biocides are common in use; however the oxidizing biocides are preferred due to their non-specificity, speed of kill, cost effectiveness, and ease of monitoring.

Many previous chemical methods make use of halogen based and in particular, chlorine-based compositions of matter. However, for various reasons, it is desirable to avoid the use of halogens in biocidal agents. Halogens tend to participate in unwanted side reactions with other chemicals in the process waters, thereby limiting the efficacy of the biocidal application and that of the applied chemical with which the halogen reacts. Also, halogen based biocides can form disinfection by-products such as AOX (adsorbable organic halide) and THMs (trihalomethanes) and in some cases cause health concerns. Finally, halogen based chemistries raise several environmental concerns due to their high reactivity and formation of disinfection by-products.

The application of any chemical microbial control technology to an industrial or potable water system is regulated and monitored by governmental agencies globally. There is an increasing demand for “greener” biocides due to increasing environmental and regulatory concerns. “Greener” biocides could encompass physical, chemical, or physio-chemical methods that provide effective microbial control and do not, or have a reduced, impact on the environment.

Peracetic acid (PAA) is an effective “green” oxidizing biocide. PAA is currently made via an equilibrium process, which requires large excesses of hydrogen peroxide and acetic acid in the presence of a strong acid catalyst. As a result, this PAA mix has significant issues related to its handling, corrosivity, odor, and is undesirable for use in many applications. As highly concentrated solutions of PAA have significant issues related to safety and the potential for explosions, it is highly desirable to use dilute solutions. This requires a large footprint for storage of a PAA solution. Additionally, PAA is prone to decompose and has issues with long term storage and stability.

PAA can be formed from a peroxygen source and a peracetic acid activator. These systems have found widespread use in laundry applications as low temperature bleaches. However, applications of this technology to other disinfection areas are limited as the PAA is formed in relatively low concentrations at a relatively slow rate and decomposes relatively quickly.

What is needed is a system that allows for an in situ method of peracetic acid generation that allows for the efficient generation of PAA from easy to handle precursors. It is desirable that the system can be mixed on site and used directly with the desired application, reducing or eliminating the need to directly handle PAA. It is desirable that the system provides stable peracetic acid for use as a biocidal control agent.

Also needed is a system that is an effective chemical method to improve the efficiency of a peracetic acid activator for use in an on-site in situ generation of a “green” oxidizing biocide that has minimal environmental impact. Indeed, microbial control represents a significant revenue stream, in utility waters and process applications. Due to implementation of new regulations, there is a market demand to move to more sustainable approaches of water treatment. There is a customer demand to reduce emissions such as AOX, and to develop new non-halogen oxidizing biocides that are considered “green.”

### SUMMARY

Disclosed herein are biocidal compounds and compositions, methods of using said compounds and compositions, and systems and processes for their preparation. In particular, disclosed herein are in situ generated biocides stabilized with a buffer.

In one embodiment, the invention is directed towards a method of microbial fouling control. The method includes forming a biocidal agent including peracetic acid by generating peracetic acid and introducing the biocidal agent into a system susceptible to biological fouling.

The peracetic acid may be generated by the reaction of at least two reactants, a peroxygen source and an acyl group donor.

In another embodiment, the invention is directed toward a system and method for preparing peracetic acid in situ, wherein the system includes a first vessel and a second vessel. The first vessel includes a mixture of an acyl group donor and a buffering agent. The second vessel includes a peroxygen source. The second vessel is configured to receive the mixture of acyl group donor and buffering agent, such that peracetic acid can be generated in situ in the second vessel. Peracetic acid can be generated in the second vessel by combining a quantity of the mixture from the first vessel with the peroxygen source in the second vessel to generate peracetic acid in situ.

In another embodiment, the invention is directed toward a system and method for preparing peracetic acid in situ, wherein the system includes a first vessel, a second vessel, and a third vessel. The first vessel includes a mixture of an acyl group donor and a buffering agent. The second vessel includes a peroxygen source. The third vessel is configured to receive the mixture of acyl group donor and buffering agent and the peroxygen source, such that peracetic acid can be generated in situ in the third vessel. Peracetic acid can be generated by combining in the third vessel a quantity of the mixture from the first vessel with a quantity of the peroxygen source from the second vessel.

In certain embodiments, depending on whether a two or three vessel system is used, the second vessel or the third vessel can be configured to deliver in situ generated peracetic acid to an industrial process water stream. The industrial process water stream can be flume water, shower water, washers, thermal processing waters, brewing liquids, fermentation liquids, CIP (clean in place) liquids, hard surface sanitization liquids, ethanol/bio-fuels process waters, pretreatment and utility waters, membrane system liquids, ion-exchange bed liquids, water used in the process/manufacture of paper, ceiling tiles, fiber board, microelectronics, E-coat liquids, electrodeposition liquids, process cleaning liquids, oil exploration services liquids, oil well completion fluids, oil well workover fluids, drilling additive fluids, oil fracturing fluids, oil and gas wells, flowline water systems, natural gas water systems, or any combination thereof.

In certain embodiments, peracetic acid may be generated in situ within an industrial process water stream itself, and remain within the industrial process water stream for the entire lifespan of the peracetic acid. In certain embodiments, at least one of the reactants may be introduced in a liquid medium.

In certain embodiments, an industrial process water stream includes a main branch and a side branch through which a portion of the industrial process water is diverted and then returned to the main branch. Depending on whether a two or three vessel system is used, peracetic acid can be added to the side branch via a direct connection to the second vessel or the third vessel, or via one or more conduits connecting the side branch to the second vessel or the third vessel. In certain embodiments, the second vessel is the side branch. In certain embodiments, the third vessel is the side branch. In certain embodiments, the water in the side branch, prior to receiving peracetic acid, passes through an apparatus that may be a water clarifier, a water softener, a chemical or non-chemical microbial control device, an on-line centrifuge, a water filter, or any combination thereof.

In certain embodiments, an industrial process water stream includes a monitor and a feeding mechanism. The monitor can be constructed and arranged to measure physical characteristics of the water process system and to output signals in response thereto. The feeding mechanism can be constructed and arranged to receive the signals and to appropriately increase, decrease, or halt the flow of peracetic acid into the industrial process water stream in response to the signals to optimally control biological fouling.

In certain embodiments, the above referenced first vessel and/or second vessel may each independently be a drum.

In certain embodiments, the above referenced second vessel or third vessel can be a pipe containing a continuous flow of the peroxygen source.

In certain embodiments, the above referenced second vessel or third vessel can be an in-line mixer, such as a static in-line mixer.

A suitable peroxygen source for use with the invention includes, but is not limited to, hydrogen peroxide, an inorganic persalt, a percarbonate salt, a persulfate salt, a perborate salt, a permanganate salt, potassium monopersulfate, a peroxysilicate, carbamide peroxide, urea hydrogen peroxide, an organic peroxide, or any combination thereof.

A suitable acyl group donor for use with the invention includes, but is not limited to, an N,N-diacyl compound, an N-acyl compound, an S-acyl compound, an O-acyl compound, TAEA, TAED, acetylsalicylic acid, pentaacetylglucose, tetraacetylglycoluril (TAGU), acetic anhydride, 1-acetyl imidazole, acetyl CoA, diacetin (glycerol diacetate), triacetin (glycerol triacetate), glycerol monoacetate, or any combination thereof.

A suitable buffer for use with the invention includes, but is not limited to, imidazole, 1-methylimidazole, benzotriazole, triethylamine, diisopropylethylamine, diisopropyl amine, piperidine, piperazine, urea, morpholine, N,N,N′N′-tetramethylethylenediamine (TMEDA), 1,8 diazabicyclo[5.4.0]undec-7-ene (DBU), bicine, 1,2,4-triazole, benzotriazole, histidine, 1,4 diazabicyclo[2.2.2]octane, guanine, caffeine, pyridine or its derivatives such as 2,6-lutidine and dipyridyl, an acylated amine such as 1-acetyl imidazole or 1-acetylindole, an acetyl ethyleneglycol, an acetyl polyethyleneglycol, a polyamine, the conjugate base of imidazole, piperidine, piperazine, diisopropylamine, morpholine, citric acid, tartaric acid, taurine, benzotriazole, histidine, guanine, glycerol, ethylene glycol, propylene gylcol, polyethylglycol, polypropyleneglycol, a polyamine, and the sodium, potassium, lithium, calcium, magnesium, or ammonium salts of carbonate, percarbonate, bicarbonate, acetate, borate, tetraborate, hydroxide, sulfate, phosphate (dibasic or tribasic) ions, or any combinations thereof.

The compounds, compositions, methods and processes are further described herein.

### DETAILED DESCRIPTION

### Definition of Terms

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of” and “consisting essentially of,” the embodiments or elements presented herein, whether explicitly set forth or not.

“Dispersion” means a fluid system comprising a solid or fluid dispersed phase, which is substantially dispersed within a liquid dispersion medium, dispersion includes but is not limited to solutions, suspensions, emulsions, azeotropes, and colloids.

“Essentially in the absence of” means that a particular material, if present at all in a particular chemical reaction, is present in that chemical reaction in an amount no greater than a trace amount, and its presence accounts for substantially none of the chemical reaction.

“Fouling” means the undesirable presence of or deposition of any organic or inorganic material in the water or on a surface.

“Liquid system” means flood waters or an environment within at least one artificial artifact, containing a substantial amount of liquid that is capable of undergoing biological fouling, it includes but is not limited to industrial liquid systems, industrial water systems, liquid process streams, industrial liquid process streams, industrial process water systems, process water applications, process waters, utility waters, water used in manufacturing, water used in industrial services, aqueous liquid streams, liquid streams containing two or more liquid phases, and any combination thereof.

“Monitor” means a device constructed and arranged to measure at least one physical or chemical characteristic and to output a signal or display in response to that measurement.

“Peroxygen producing chemical” means a composition of matter that contains two or more oxygen atoms in the form of an oxygen-oxygen bond and that induce a higher oxidation state in another composition of matter, peroxygen producing chemical includes but is not limited to: hydrogen peroxide, percarbonate salts, persulfate salts, perborate salts, permanganate salts, carbamide peroxide, and alkyl peroxides such as tert-butyl hydroperoxide and potassium monopersulfate.

“Primary Determinant” means a reactant in a chemical reaction whose presence is not necessary for the reaction to occur but whose presence results in a dramatic (more than 50%) increase in the reaction rate and/or yield.

“TAED” means tetraacetylethylenediamine.

“TAEA” means triacetylethanolamine.

### Biocides

In at least one embodiment, a peracetic acid containing biocide is generated in situ within a volume and added to an industrial process system volume susceptible to biofouling. The peracetic acid is generated by the reaction within the volume of a peroxygen source with an activator, such as an acyl group donor.

In at least one embodiment, the formation of the peracetic acid occurs within a pH range of between 5 and 8. In another embodiment, the formation of the peracetic acid occurs within a pH range of between 7 and 8. Preferably, a pH adjusting and/or buffering agent is used to maintain the pH within this range. The solution pH impacts both the formation and self-decomposition of peracetic acid. Alkaline conditions favor rapid formation and self-decomposition. Conversely acidic conditions hinder formation and self-decomposition. A balance must be achieved to both rapidly form peracetic acid and hinder its self-decomposition. In the disclosed invention, the buffering agent aids in the peracetic acid formation. As the peracetic acid is formed, the pH of the solution decreases to a value between 5 and 7. The lower pH inhibits peracetic acid self-decomposition. In addition, a neutral or near neutral pH range limits issues related to corrosion or pitting due to highly acidic equilibrium PAA. Providing control of the pH range allows for a longer lifetime of PAA solution, allowing for an improved effective yield while increasing the application dose.

Peracetic acid is known as an ideal biocidal agent because it has a high oxidizing potential, it is not deactivated by catalase and similar enzymes unlike hydrogen peroxide, and it breaks down into environmentally friendly residues (acetic acid and hydrogen peroxide). Peracetic acid however has a relatively short shelf life under certain pH conditions. After 10 hours or so, peracetic acid degrades and its usefulness as a biocidal agent is limited. The commercially available peracetic acid products exist as an equilibrium mixture with hydrogen peroxide and acetic acid in the presence of stabilizers and acid catalysts, such as sulfuric acid, in order to stabilize and improve the composition's shelf life. However, the composition of equilibrium PAA presents several other issues upon application, such as those of safety, handling, odor, and corrosivity. As a result, the ability to generate peracetic acid in situ greatly aids in the logistics and effectiveness of a peracetic acid based biocidal agent. Furthermore, in situ generation allows the peracetic acid to be applied effectively to a site for up to its entire lifespan.

This ability of peroxygen sources to generate biocides in situ under standard conditions is quite unexpected when one looks at previous uses of peroxygen sources. Peroxygen sources such as hydrogen peroxide are commonly used as biocides, but are required at higher concentrations for effectiveness. In addition, peroxygen sources are commonly used in laundry detergent formulations as a more versatile alternative to chlorine bleaches. In such applications, peroxygen sources are often used with additives such as TAED (tetraacetylethylenediamine) and NOBS (nonanoyloxybenzene sulfonate) to facilitate the production and efficacy of bleaching agents. In the laundry context they are only expected to function in an alkaline environment of at least pH 9.

In at least one embodiment, the invention is distinct from and different from the chemistries used in laundry applications. In at least one embodiment, the generated peracetic acid is a dispersion.

Peracetic acid generation from TAED, as may occur in laundry applications, is aided by the use of surfactants. Surfactants facilitate the dissolution of TAED into water, but the dissolution is slow and this creates a time-release effect for peracetic acid generation. Such time-released peracetic acid generation may be unacceptable for many biocidal applications, as the peracetic acid concentration may be too low to effectively act as a biocide in the selected environment.

Laundry applications are carried out in high pH environments (9-11) to facilitate bleaching of textile materials. The solution pH impacts both the formation and self-decomposition of peracetic acid. Alkaline conditions favor rapid formation and self-decomposition. Conversely acidic conditions hinder formation and self-decomposition. In contrast, the invention operates in a lower range (4-11 and preferably 5-8). The lower pH range works to stabilize the peracetic acid. Laundry applications are single container reactions and do not have the same runability and application issues as industrial systems, so a solid such as TAED is acceptable in laundry applications.

Accordingly, in at least one embodiment, the acyl group donor is a water soluble compound, such as triacetylethanolamine. TAEA is a liquid and utilizes surfactants to allow the chemistry to flow through a complex system or apparatus. Laundry applications are of short duration (15-45 minutes) because longer exposure to the agents can damage the textiles and is unnecessary because the textiles cannot get any cleaner with more time. Laundry applications are single container applications where solid chemistries are easily applied as part of a solid detergent formulation. There is however a lack of commercially available technology where in situ production of peracetic acid is conducted in the liquid state using liquid precursors. In at least one embodiment the peracetic acid solution generated is stable on the order of hours to days. In laundry applications, a carbonate material (such as sodium carbonate) is the primary determinant of the acid yield. In at least one embodiment, the maintaining of the pH at a level which stabilizes the generated peracetic acid is the primary determinant of yield. If the pH gets too high the peracetic acid breaks down dramatically reducing yield and if the pH is too low the reaction rate slows dramatically reducing yield. Within at least one embodiment, the pH range inhibits peracetic acid self-decomposition.

In at least one embodiment, a side stream of the process water is diverted, the biocidal agent is added to the side stream, and the side stream is re-introduced to the process water stream. In at least one embodiment, the side stream is run through a water conditioning system, such as a clarifier or a filtration system, before the biocidal agent is added. In at least one embodiment, a monitoring system is present in the water process system which measures physical or chemical characteristics indicating changes in biocide concentrations, degree of biofouling, flow rate, and the like, and any combination thereof.

In at least one embodiment, this technology would be applicable to any process or utility liquid system where microorganisms are known to grow and are an issue, and biocides are added. Examples of some industrial process water systems where the method of this invention could be applied are in process water applications (e.g., flume water, shower water, washers, thermal processing waters, brewing, fermentation, CIP (clean in place), hard surface sanitization, etc.), ethanol/bio-fuels process waters, pretreatment and utility waters (e.g., membrane systems, ion-exchange beds), water used in the process/manufacture of paper, ceiling tiles, fiber board, microelectronics, E-coat or electrodeposition applications, process cleaning, oil exploration and energy services (e.g., completion and workover fluids, drilling additive fluids, fracturing fluids, flood waters, etc; oil fields—oil and gas wells/flowline, water systems, gas systems, etc.), and in particular water systems where the installed process equipment exhibits lowered compatibility to halogenated biocides.

In at least one embodiment, the mechanism feeding the biocidal agents and or its constituents are automated, constructed, and arranged to appropriately feed or cut off the feed of materials into the water process stream in response to receiving signals from the monitors. In at least one embodiment, the applied biocidal dosage is determined according to oxidation-reduction potential (ORP) techniques. In at least one embodiment, the rate of biofouling is known and the feed rate of biocidal agent is timed to be optimal against that rate. Such embodiments are particularly effective when the particular species or ecology of the biological contaminant is identified and the growth rate is known for the given environment.

In at least one embodiment, the system into which the biocidal agent is introduced is a system which excludes laundry washing, laundry bleaching, textile washing, and/or textile bleaching systems. In at least one embodiment, the systems contemplated into which the biocidal agent is introduced includes laundry washing, laundry bleaching, textile washing, and/or textile bleaching systems.

Peroxygen sources that can be used with the present invention include, but are not limited to, hydrogen peroxide, an inorganic persalt, a percarbonate salt, a persulfate salt, a perborate salt, a permanganate salt, potassium monopersulfate, a peroxysilicate, carbamide peroxide, urea hydrogen peroxide, an organic peroxide, or any combination thereof.

Acyl group donors that can be used with present invention include, but are not limited to, an N,N-diacyl compound, an N-acyl compound, an S-acyl compound, an O-acyl compound, triacetylethanolamine, tetraacetylethylenediamine, acetylsalicylic acid, pentaacetylglucose, tetraacetylglycoluril, acetic anhydride, 1-acetyl imidazole, acetyl CoA, diacetin (glycerol diacetate), triacetin (glycerol triacetate), glycerol monoacetate, or any combination thereof.

Buffers that can be used with the present invention include, but are not limited to, imidazole, 1-methylimidazole, benzotriazole, triethylamine, diisopropylethylamine, diisopropyl amine, piperidine, piperazine, urea, morpholine, N,N,N′N′-tetramethylethylenediamine (TMEDA), 1,8 diazabicyclo[5.4.0]undec-7-ene (DBU), bicine, 1,2,4-triazole, benzotriazole, histidine, 1,4 diazabicyclo[2.2.2]octane, guanine, caffeine, pyridine or its derivatives such as 2,6-lutidine and dipyridyl, acylated amines such as 1-acetyl imidazole or 1-acetylindole, acetyl ethyleneglycols, acetyl polyethyleneglycols, polyamines, the conjugate base of imidazole, piperidine, piperazine, diisopropylamine, morpholine, citric acid, tartaric acid, taurine, benzotriazole, histidine, guanine, glycerol, ethylene glycol, propylene gylcol, polyethylglycol, polypropyleneglycol, polyamines, and the sodium, potassium, lithium, calcium, magnesium, or ammonium salts of carbonate, percarbonate, bicarbonate, acetate, borate, tetraborate, hydroxide, sulfate, phosphate (dibasic or tribasic) ions, and combinations thereof.

In at least one embodiment, the buffer can function both as a base to control the pH and as an peracetic acid activator. For example, the acylated amines, such as 1-acetyl imidazole, can function both as bases to control the pH, and as peracetic acid activators.

In at least one embodiment, the buffer can function both as a base to control the pH and as a corrosion inhibitor. For example, imidazole and/or benzotriazole can function both as buffers and corrosion inhibitors in the industrial system they are placed within. Suitable masked bases include 1-acetyl imidazole.

In at least one embodiment, the buffer may serve as an acetyl transfer agent. It can remove an acetyl group from the acyl group donor making it more susceptible to attack by hydrogen peroxide to form peracetic acid. These buffers are generally bases having a nitrogen- or sulfur-containing heterocycle, for example 1-acetyl imidazole. As a base it is capable of absorbing a proton and acting as a buffer.

The use of a suitable, soluble buffer that is a base may also prevent degradation or hydrolysis of the PAA activator. This increases the shelf life of an activator solution.

Biocides within the scope of the present invention include, but are not limited to, peracetic acid and hydrogen peroxide. In at least one embodiment, the biocidal agent further comprises one or more items selected from the group consisting of: surfactants, chelants, dispersants, emulsifiers, salts, and freezing point depressants, or any combination thereof.

### Systems and Methods

In addition to the aforementioned advantages, the present invention allows users to avoid the need for multiple chemical feedstocks. The invention can be supplied by adding contents from as few as 2 chemical storage containers into a target object. The use of a soluble buffer, preferably 1-acetyl imidazole, allows for the development of an in situ generation system that uses two total solutions. In turn, this allows for a simpler operational set up and is an improvement over the current solution systems.

Additionally, a two solution system has a reduced physical footprint, allowing for use in areas that have limited floor space or have a limited load capacity. Previous systems require storage of the chemical solutions in up to four different containers, in order to prevent degradation of the active chemicals, and may require the use of solubilizing agents or surfactants to improve the performance of the disinfectant. In at least one embodiment of the current invention, the system is not reliant upon the use of a highly reactive hydroperoxycarbonate species to generate peracetic acid.

In some embodiments, peracetic acid can be generated by combining the activator, buffer, and hydrogen peroxide through a series of pipes into a day tank or in-line. Alternatively the activator and buffer can be formulated into one drum then mixed with hydrogen peroxide. Preferably, the buffer is not added to the peroxide as a single drum. Preferably, the activator is always added to the peroxide to keep the peroxide in excess to avoid forming potentially explosive diacetylperoxide. The peracetic acid could then be introduced to the water system as a slug feed or continuous dose.

### Two Vessel System and Method

In some embodiments, the invention is directed toward a system and method for preparing peracetic acid in situ, wherein the system includes a first vessel and a second vessel. The first vessel includes a mixture of an acyl group donor and a buffering agent, as described above. The second vessel includes a peroxygen source, as described above. The second vessel is configured to receive the mixture of acyl group donor and buffering agent, such that peracetic acid can be generated in situ in the second vessel. Peracetic acid can be generated in the second vessel by combining a quantity of the mixture from the first vessel with the peroxygen source in the second vessel to generate peracetic acid in situ.

In certain embodiments, the second vessel can be configured to deliver in situ generated peracetic acid to an industrial process water stream. The industrial process water stream can be flume water, shower water, washers, thermal processing waters, brewing liquids, fermentation liquids, CIP (clean in place) liquids, hard surface sanitization liquids, ethanol/bio-fuels process waters, pretreatment and utility waters, membrane system liquids, ion-exchange bed liquids, water used in the process/manufacture of paper, ceiling tiles, fiber board, or microelectronics, E-coat liquids, electrodeposition liquids, process cleaning liquids, oil exploration services liquids, oil well completion fluids, oil well workover fluids, drilling additive fluids, oil fracturing fluids, oil and gas wells, flowline water systems, natural gas water systems, or any combination thereof.

In certain embodiments, the peracetic acid may be generated in situ within the industrial process water stream itself, and remain within the industrial process water stream for the entire lifespan of the peracetic acid. In certain embodiments, at least one of the reactants may be introduced in a liquid medium.

In certain embodiments, the industrial process water stream includes a main branch and a side branch through which a portion of the industrial process water is diverted and then returned to the main branch. Peracetic acid can be added to the side branch via a direct connection to the second vessel, or via one or more conduits connecting the side branch to the second vessel. In certain embodiments, the second vessel is the side branch. In certain embodiments, the water in the side branch, prior to receiving peracetic acid, passes through an apparatus that may be a water clarifier, a water softener, a chemical or non-chemical microbial control device, an on-line centrifuge, a water filter, or any combination thereof.

In certain embodiments, the industrial process water stream includes a monitor and a feeding mechanism. The monitor can be constructed and arranged to measure physical characteristics of the water process system and to output signals in response thereto. The feeding mechanism can be constructed and arranged to receive the signals and to appropriately increase, decrease, or halt the flow of peracetic acid into the industrial process water stream in response to the signals to optimally control biological fouling.

In certain embodiments, the first vessel and/or the second vessel may each independently be a drum.

In certain embodiments, the second vessel may be a pipe containing a continuous flow of the peroxygen source, wherein the second vessel is configured to receive the mixture of acyl group donor and buffering agent from the first vessel, such that peracetic acid can be generated in situ in the second vessel.

In certain embodiments, the second vessel may be an in-line mixer containing a continuous flow of the peroxygen source, wherein the second vessel is configured to receive a flow, optionally a continuous flow, of the mixture of acyl group donor and buffering agent from the first vessel. The in-line mixer may be, for example, a static in-line mixer or a high shear in-line mixer. Accordingly, the second vessel may an in-line mixer containing a continuous flow of the peroxygen source and a continuous flow of the mixture of acyl group donor and buffering agent from the first vessel.

In certain embodiments, generation of peracetic acid in the second vessel occurs at a pH of between 4 and 11, or at a pH of between 5 and 8.

In certain embodiments, the second vessel is a vessel other than a vessel for laundry washing, laundry bleaching, textile washing, and/or textile bleaching.

### Three Vessel System and Method

In some embodiments, the invention is directed toward a system and method for preparing peracetic acid in situ, wherein the system includes a first vessel, a second vessel, and a third vessel. The first vessel includes a mixture of an acyl group donor and a buffering agent, as described above. The second vessel includes a peroxygen source, as described above. The third vessel is configured to receive the mixture of acyl group donor and buffering agent and the peroxygen source, such that peracetic acid can be generated in situ in the third vessel. Peracetic acid can be generated by combining in the third vessel a quantity of the mixture from the first vessel with a quantity of the peroxygen source from the second vessel.

In certain embodiments, the third vessel can be configured to deliver in situ generated peracetic acid to an industrial process water stream. The industrial process water stream can be flume water, shower water, washers, thermal processing waters, brewing liquids, fermentation liquids, CIP (clean in place) liquids, hard surface sanitization liquids, ethanol/bio-fuels process waters, pretreatment and utility waters, membrane system liquids, ion-exchange bed liquids, water used in the process/manufacture of paper, ceiling tiles, fiber board, or microelectronics, E-coat liquids, electrodeposition liquids, process cleaning liquids, oil exploration services liquids, oil well completion fluids, oil well workover fluids, drilling additive fluids, oil fracturing fluids, oil and gas wells, flowline water systems, natural gas water systems, or any combination thereof.

In certain embodiments, the peracetic acid may be generated in situ within the industrial process water stream itself, and remain within the industrial process water stream for the entire lifespan of the peracetic acid. In certain embodiments, at least one of the reactants may be introduced in a liquid medium.

In certain embodiments, the industrial process water stream includes a main branch and a side branch through which a portion of the industrial process water is diverted and then returned to the main branch. The peracetic acid can be added to the side branch from the third vessel via a direct connection to the third vessel, or via one or more conduits connecting the side branch to the third vessel. In certain embodiments, the third vessel is the side branch. In certain embodiments, the water in the side branch, prior to receiving peracetic acid, passes through an apparatus that may be a water clarifier, a water softener, a chemical or non-chemical microbial control device, an on-line centrifuge, a water filter, or any combination thereof.

In certain embodiments, the industrial process water stream includes a monitor and a feeding mechanism. The monitor can be constructed and arranged to measure physical characteristics of the water process system and to output signals in response thereto. The feeding mechanism can be constructed and arranged to receive the signals and to appropriately increase, decrease, or halt the flow of peracetic acid into the industrial process water stream in response to the signals to optimally control biological fouling.

In certain embodiments, the first vessel and/or the second vessel may each independently be a drum, and the third vessel can be a pipe containing a continuous flow of the peroxygen source received from the second vessel. Peracetic acid can be generated in situ in the third vessel upon receiving the mixture of acyl group donor and buffering agent from the first vessel.

In certain embodiments, the third vessel may be an in-line mixer containing a continuous flow of the peroxygen source from the second vessel, wherein the third vessel is configured to receive a flow, optionally a continuous flow, of the mixture of acyl group donor and buffering agent from the first vessel. The in-line mixer may be, for example, a static in-line mixer or a high shear in-line mixer. Accordingly, the third vessel may an in-line mixer containing a continuous flow of the peroxygen source from the second vessel and a continuous flow of the mixture of acyl group donor and buffering agent from the first vessel.

In certain embodiments, generation of peracetic acid in the third vessel occurs at a pH of between 4 and 11, or at a pH of between 5 and 8.

In certain embodiments, the third vessel is a vessel other than a vessel for laundry washing, laundry bleaching, textile washing, and/or textile bleaching.

In certain embodiments, the aforementioned first, second, and third vessels of the invention may each independently include additional agents to serve as one or more additional components of the biocidal composition. For example, the first vessel may include a surfactant, chelant, dispersant, emulsifier, salt, freezing point depressant, or any combination thereof

### EXAMPLES

The foregoing may be better understood by reference to the following example, which is presented for purposes of illustration and is not intended to limit the scope of the invention.

### Example 1

1-acetyl imidazole was dissolved in triacetylethanolamine with stirring. Hydrogen peroxide was added with stirring. After 15 minutes at room temperature the concentration of peracetic acid was determined using standard laboratory titration equipment. Hydrogen peroxide (17%) was purchased from Solvay. Experimental grade 1-acetyl imidazole was purchased from Sigma-Aldrich.

The assay for peracetic acid is an iodometric titration. A beaker was charged with crushed ice (100 mL). Glacial acetic acid (10 mL) was added to the ice followed by potassium iodide (2.5 mL, 20%). An aliquot of the reaction mixture containing peracetic acid, typically 500 to 1000 μL, was added to the iced solution. A dark yellow or brown color will form if peracetic acid is present. With vigorous stirring, the iced solution was rapidly titrated with sodium thiosulfate (0.1 M) until clear. The titrant volume was used to calculate the concentration of peracetic acid in millimoles using the following equation:

$$x mL {Na}_{2} {S}_{2} {O}_{3} \times 0.1 M {Na}_{2} {S}_{2} {O}_{3} \times \frac{1 m mol PAA}{2 mmol {Na}_{2} {S}_{2} {O}_{3}} \div y mL Reaction mixture = z mmol PAA$$

Table 1 demonstrates that the yield of peracetic acid from the combination of a hydrogen peroxide peroxygen source with a TAEA activator is unexpectedly high. The table indicates how much of a molar excess of hydrogen peroxide per mol of TAEA activator is required to produce the best yields. The yield % reflects the percentage of acetyl source converted into peracetic acid. The reaction was performed in the presence of various amounts of 1-acetyl imidazole.

TABLE 1
Peracetic acid yields from hydrogen peroxide and an acyl group donor
(triacetylethanolamine)

| | Hydrogen | |
|---|---|---|
| 1-acetyl | peroxide | |
| imidazole | molar | Peracetic |
| wt % | excess | acid yield |
| 0.0% | 0.99 | 7% |
| 0.0% | 2.31 | 8% |
| 1.0% | 9.07 | 33% |
| 1.0% | 7.18 | 19% |
| 1.0% | 4.91 | 13% |
| 1.0% | 1.99 | 16% |
| 1.0% | 1.49 | 38% |
| 1.0% | 1.00 | 22% |
| 2.0% | 8.93 | 55% |
| 2.0% | 7.07 | 26% |
| 2.0% | 7.07 | 26% |
| 2.0% | 4.84 | 19% |
| 3.0% | 9.15 | 93% |
| 3.0% | 6.96 | 41% |
| 3.0% | 5.13 | 29% |
| 3.1% | 3.86 | 85% |
| 3.1% | 2.89 | 72% |
| 3.1% | 1.93 | 62% |
| 3.1% | 1.45 | 56% |
| 3.1% | 1.45 | 57% |
| 3.1% | 1.45 | 59% |
| 3.1% | 1.45 | 60% |
| 3.1% | 1.45 | 63% |
| 3.1% | 0.96 | 53% |
| 4.0% | 3.80 | 91% |
| 4.0% | 2.85 | 77% |
| 5.0% | 3.91 | 60% |
| 5.0% | 3.73 | 61% |
| 5.0% | 3.73 | 78% |
| 5.1% | 3.74 | 90% |
| 5.1% | 2.80 | 79% |
| 5.1% | 1.40 | 48% |
| 5.7% | 2.12 | 83% |
| 5.7% | 0.91 | 60% |
| 6.1% | 2.76 | 90% |
| 6.1% | 1.84 | 81% |
| 6.1% | 0.92 | 56% |
| 7.1% | 2.72 | 95% |
| 7.1% | 1.81 | 78% |
| 7.1% | 1.81 | 80% |
| 7.1% | 0.91 | 56% |
| 8.1% | 2.68 | 96% |
| 8.1% | 1.79 | 81% |
| 8.1% | 0.89 | 61% |
| 11.4% | 1.95 | 81% |
| 11.4% | 0.84 | 56% |

Any ranges given either in absolute terms or in approximate terms are intended to encompass both, and any definitions used herein are intended to be clarifying and not limiting. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges (including all fractional and whole values) subsumed therein.

Furthermore, the invention encompasses any and all possible combinations of some or all of the various embodiments described herein. Any and all patents, patent applications, scientific papers, and other references cited in this application, as well as any references cited therein, are hereby incorporated by reference in their entirety.

What is claimed is:

1. A system for preparing peracetic acid in situ, comprising:
a first vessel comprising a mixture of an acyl group donor and a buffering agent; and
a second vessel comprising a peroxygen source;
wherein the second vessel is configured to receive the mixture of acyl group donor and buffering agent, such that peracetic acid can be generated in situ in the second vessel.

2. The system of claim 1, wherein the second vessel is configured to deliver in situ generated peracetic acid to an industrial process water stream, wherein the industrial process water stream is selected from the group consisting of flume water, shower water, washers, thermal processing waters, brewing liquids, fermentation liquids, CIP (clean in place) liquids, hard surface sanitization liquids, ethanol/bio-fuels process waters, pretreatment and utility waters, membrane system liquids, ion-exchange bed liquids, water used in the process/manufacture of paper, ceiling tiles, fiber board, or microelectronics, E-coat liquids, electrodeposition liquids, process cleaning liquids, oil exploration services liquids, oil well completion fluids, oil well workover fluids, drilling additive fluids, oil fracturing fluids, oil and gas wells, flowline water systems, natural gas water systems, and any combination thereof.

3. The system of claim 2, wherein the industrial process water stream comprises a main branch and a side branch through which a portion of the industrial process water is diverted and then returned to the main branch, wherein at least peracetic acid is added to the side branch via direct connection to the second vessel, or via one or more conduits connecting the side branch to the second vessel.

4. The system of claim 1, wherein the first vessel is a drum; the second vessel is a drum; or both the first and the second vessel are each a drum.

5. The system of claim 1, wherein the second vessel is a pipe comprising a continuous flow of the peroxygen source.

6. The system of claim 3, wherein the second vessel is the side branch.

7. The system of claim 1, wherein the peroxygen source is selected from the group consisting of: hydrogen peroxide, inorganic persalts, percarbonate salts, persulfate salts, perborate salts, permanganate salts, potassium monopersulfate, peroxysilicates, carbamide peroxide, urea hydrogen peroxide, organic peroxides, and any combination thereof.

8. The system of claim 1, wherein the acyl group donor is selected from the group consisting of: an N,N-diacyl compound, an N-acyl compound, an S-acyl compound, an O-acyl compound, TAEA, TAED, acetylsalicylic acid, pentaacetylglucose, tetraacetylglycoluril (TAGU), acetic anhydride, 1-acetyl imidazole, acetyl CoA, diacetin (glycerol diacetate), triacetin (glycerol triacetate), glycerol monoacetate, and any combination thereof.

9. The system of claim 1, wherein generation of peracetic acid in the second vessel occurs at a pH of between 4 and 11.

10. The system of claim 1, wherein the acyl group donor is triacetylethanolamine, the buffering agent is 1-acetyl imidazole or imidazole, and the peroxygen source is hydrogen peroxide.

11. The system of claim 1, wherein the second vessel is a vessel other than a vessel for laundry washing, laundry bleaching, textile washing, and/or textile bleaching.

12. A method for preparing peracetic acid in situ, comprising:
providing a first vessel comprising a mixture of an acyl group donor and a buffering agent;
providing a second vessel comprising a peroxygen source; and
combining a quantity of the mixture from the first vessel with the peroxygen source in the second vessel to generate peracetic acid in situ.

13. The method of claim 12, wherein at least a portion of the generated peracetic acid is delivered from the second vessel to an industrial process water stream, wherein the industrial process water stream is selected from the group consisting of flume water, shower water, washers, thermal processing waters, brewing liquids, fermentation liquids, CIP (clean in place) liquids, hard surface sanitization liquids, ethanol/bio-fuels process waters, pretreatment and utility waters, membrane system liquids, ion-exchange bed liquids, water used in the process/manufacture of paper, ceiling tiles, fiber board, or microelectronics, E-coat liquids, electrodeposition liquids, process cleaning liquids, oil exploration services liquids, oil well completion fluids, oil well workover fluids, drilling additive fluids, oil fracturing fluids, oil and gas wells, flowline water systems, natural gas water systems, and any combination thereof.

14. The method of claim 13, wherein the industrial process water stream comprises a main branch and a side branch through which a portion of the industrial process water is diverted and then returned to the main branch, wherein at least peracetic acid is added to the side branch via direct connection to the second vessel, or via one or more conduits connecting the side branch to the second vessel.

15. The method of claim 12, wherein the first vessel is a drum.

16. The method of claim 12, wherein the second vessel is a drum.

17. The method of claim 12, wherein the second vessel is a pipe comprising a continuous flow of the peroxygen source.

18. The method of claim 14, wherein the second vessel is the side branch.

19. The method of claim 12, wherein generation of peracetic acid in the second vessel occurs at a pH of between 4 and 11.

20. The method of claim 12, wherein the acyl group donor is triacetylethanolamine, the buffering agent is 1-acetyl imidazole or imidazole, and the peroxygen source is hydrogen peroxide.
